(19) United States
(12) Patent Application Publication
(10) Pub. No.: US 2024/0367171 A1
(43) Pub. Date: Nov. 7, 2024

(54) MICROFLUIDIC DEVICE

(71) Applicant: Hewlett-Packard Development Company, L.P., Spring, TX (US)

(72) Inventors: Michael Cumbie, Corvallis, OR (US); Viktor Shkolnikov, Palo Alto, CA (US)

(73) Assignee: Hewlett-Packard Development Company, L.P., Spring, TX (US)

(21) Appl. No.: 18/557,564

(22) PCT Filed: Apr. 30, 2021
(86) PCT No.: PCT/US2021/030081

§ 371 Date: Oct. 26, 2023

(51) Int. Cl. B01L 3/00; B01L 7/00

(52) U.S. Cl. CPC B01L 3/502761; B01L 3/502738; B01L 7/52; B01L 2200/16; B01L 2300/0816; B01L 2300/087; B01L 2400/0677

(57) ABSTRACT

A microfluidic device is described. The device comprises a reaction chamber, wherein the reaction chamber comprises at least one reaction reagent disposed on at least one inner surface of the reaction chamber. A heater is also provided. A thermally dissolvable or degradable or thermally degradable film is applied to the at least one inner surface of the reaction chamber on which the reaction reagent is disposed. Also described is a PCR apparatus and a method of performing PCR.

### BACKGROUND

Microfluidic devices are used to transport, separate, mix or process fluids on a microscale. Microfluidic devices typically comprise a pattern of connected moulded or engraved microchannels which are incorporated into a microfluidic chip. The design of a microfluidic device can include passive fluid control using capillary forces or active fluid control can also be achieved by using microcomponents such as micropumps and microvalves. Microfluidic devices can also be used to perform multi-step reactions and are often referred to as “lab on chip” devices.

### BRIEF DESCRIPTION OF THE DRAWINGS

FIG. 1 is a plan view of an example microfluidic device of the present disclosure.

FIG. 2 is a side view of the microfluidic device of FIG. 1.

FIG. 3A is a plan view of the base of a reaction chamber of a microfluidic device of the present disclosure with disposed reaction reagents individually isolated or covered by a thermally dissolvable or degradable film.

FIG. 3B is a plan view of the base of a reaction chamber of a microfluidic device of the present disclosure with disposed reaction reagents individually isolated or covered by a thermally dissolvable or degradable film and barriers shown in a parallel design.

FIG. 3C is a plan view of the base of a reaction chamber of a microfluidic device of the present disclosure with disposed reaction reagents individually isolated or covered by a thermally dissolvable or degradable film and a barrier shown in a serpentine design.

FIG. 4A is a plan view of the base of a reaction chamber of a microfluidic device of the present disclosure with disposed reaction reagents and a thermally dissolvable or degradable film applied to protect the reaction reagents.

FIG. 4B is similar to FIG. 3A and additionally includes parallel barriers.

FIG. 4C is similar to FIG. 3A and additionally includes a serpentine barrier.

### DETAILED DESCRIPTION

Before particular embodiments of the present method and other aspects are disclosed and described, it is to be understood that the present device, apparatus, method and other aspects are not limited to the particular process and materials disclosed herein as such may vary to some degree. It is also to be understood that the terminology used herein is used for the purpose of describing particular embodiments only and is not intended to be limiting, as the scope of the present method and other aspects will be defined only by the appended claims and equivalents thereof.

In the present specification, and in the appended claims, the following terminology will be used:

The singular forms “a”, “an” and “the” include plural referents unless the context clearly dictates otherwise. Thus, for example, reference to “a sensor” includes reference to one or more of such sensors.

The terms “about” and “approximately” when referring to a numerical value or range is intended to encompass the values resulting from experimental error that can occur when taking and/or making measurements.

Concentrations, amounts, and other numerical data may be presented herein in a range format. It is to be understood that such a range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight range of approximately 1 wt. % to approximately 20 wt. % should be interpreted to include not only the explicitly recited concentration limits of 1 wt. % to approximately 20 wt. %, but also to include individual concentrations such as 2 wt. %, 3 wt. %, 4 wt. %, and sub-ranges such as 5 wt. % to 10 wt. %, 10 wt. % to 20 wt. %, etc.

As used herein, the abbreviations “PCR”, “dNTPs” and “primers” refer to the “Polymerase Chain Reaction” and its components. Specifically, the term “dNTP” refers to the 2′-deoxynucleotide triphosphates used in PCR. The four standard dNTPs are 2′-deoxyadenosine 5′-triphosphate, 2′-deoxyguanosine 5′-triphosphate, 2′-deoxycytosine 5′-triphosphate and thymidine 5′-triphosphate (already lacking a 2′-hydroxyl), though modified dNTPs incorporating labels or reporter molecules, or reactive moieties may also be used.

As used herein, the term “primer” refers to a short single stranded nucleic acid, typically an oligodeoxynucleotide (also referred to as an oligonucleotide herein), of about 15 to 30 nucleotides in length. A primer is designed to base pair in a specific or complementary manner to a nucleic acid sequence of interest, and so is considered specific to that nucleic acid. DNA is directional, with the 3′ end of one strand forming base pairs with the 5′-end of the counter strand and a primer is usually designed so that its 5′-end base pairs to the 3′-end of the nucleic acid of interest so that DNA synthesis (which occurs in a 5′ to 3′ direction) to elongate the primer can occur.

As used herein, the terms “oligonucleotide pair”, “oligonucleotide primer pair” and “primer pair” refer to a set of two oligonucleotides that can serve as forward and reverse primers for a nucleic acid of interest. As both strands are copied and amplified in a PCR reaction, each strand requires a primer: the forward primer attaches to the start codon of the template DNA strand (the anti-sense strand), while the reverse primer attaches to the stop codon of the complementary strand of DNA (the sense strand). The 5′-end of each primer binds to the 3′-end of the complementary DNA strand of the nucleic acid of interest.

As used herein, the term “nucleic acid of interest”, or “target”, refers to a polynucleotide sequence, typically of at least one hundred, two hundred, three hundred, four hundred, five hundred or up to one thousand nucleotides in length. The polynucleotide sequence may be specific to a particular organism such as a pathogen, or may be suspected of having a particular mutation along its length, and will encode a particular polypeptide or protein, or mutant form thereof. For example, the polynucleotide sequence may encode the spike protein of SARS-COV-2, or may encode a mutant form of the epidermal growth factor receptor (EGFR) the presence or absence of which renders a patient more or less likely to respond well to cancer treatments such as erlotinib or gefitinib.

As used herein, the term “thermally dissolvable or degradable film” refers to a film of material, for example a polymeric film, which isolates a reaction reagent, to protect it from premature, or unwanted reaction. The thermally dissolvable or degradable film is inert to any storage condition, and to any aqueous reaction solvent or solution, at room temperature. The thermally dissolvable or degradable film will dissolve and/or degrade under conditions of elevated temperature when in contact with an aqueous reaction solvent or solution, as described herein.

In the following description, for purposes of explanation, numerous specific details are set forth in order to provide a thorough understanding of the present apparatus and methods. It will be apparent, however, to one skilled in the art, that the present apparatus and methods maybe practiced without these specific details. Reference in the specification to “one example” or “an example” means that a particular feature, structure, or characteristic described in connection with the example is included in at least one example. The appearance of the phrase “in one example” in various places in the specification are not necessarily all referring to the same example.

Unless otherwise stated, any feature described herein can be combined with any aspect or any other feature described herein.

The Polymerase Chain Reaction (PCR) is used to amplify specific nucleic acid sequences of interest and detect their presence in a sample. PCR can be used for many different applications, including quantification of gene expression, patient genotyping and also as a diagnostic tool to identify the presence of one or more pathogens, for example bacteria or viruses in a sample from a patient by amplifying and detecting nucleic acid sequences that are specific to a particular pathogen. Personalised medicine requires genotyping using PCR in which the detection of one or more biomarkers, for example specific mutations, may influence clinical decisions on the nature or type of medical intervention.

Regardless of end application, PCR subjects a sample to multiple rounds of thermocycling in the presence of an enzyme capable of elongating nucleic acid strands, for example a polymerase. Polymerases catalyse the reaction between a deoxynucleotide triphosphate and a DNA strand, producing an elongated DNA strand bearing one more nucleotide (from the deoxynucleotide triphosphate), and pyrophosphate as a by-product, Examples of polymerases used in PCR are thermostable polymerases such as Taq polymerase (from Thermus aquaticus), Pfu polymerase (from Pyrococcus furiosus), and Bst polymerase (from Bacillus stearothermophilus). The DNA strand that is elongated in PCR is usually in the form of an oligonucleotide primer specific to a target nucleic acid sequence of interest, which is elongated using a mixture of deoxyribonucleotide triphosphates (dNTPs). For full synthesis of a standard DNA strand, four dNTPs corresponding to the four nucleobases found in DNA (adenine, guanosine, thymine and cytosine) are required: 2′-deoxyadenosine 5′-triphosphate, 2′-deoxyguanosine 5′-triphosphate, 2′-deoxycytosine 5′-triphosphate and thymidine 5′-triphosphate.

The amplification products (amplicons) are detected optically, typically using fluorescent reporters. Fluorescent reporter molecules used in PCR include non-specific fluorescent dyes, such as SYBR Green I, which has a distinct emission spectrum when intercalated into any double-stranded DNA, leading to an increase in fluorescence as more double-stranded DNA is produced. Other suitable reporter molecules include target-specific fluorescent reporter molecules, such as the TaqMan hydrolysis probes of target-specific nucleic acids labelled with fluorescent reporter and quencher, with the probe being hydrolyzed by the exonuclease activity of the Taq polymerase, releasing the reporter from the quencher and again leading to an increase in fluorescence. Reporter molecules may also be linked to a primer to be used in the PCR amplification, such as in the Scorpion® system, a single-stranded bi-labeled fluorescent probe sequence forming a hairpin-loop conformation with a 5′ end reporter and an internal quencher directly linked to the 5′ end of a primer via a blocker (which prevents the polymerase from extending the primer). In the beginning, the polymerase extends the primer and synthesizes the complementary strand of the specific target sequence. During the next cycle, the hairpin-loop unfolds and the loop-region of the probe hybridizes intramolecularly to the newly synthesized target sequence. Now that the reporter is no longer in close proximity to the quencher, fluorescence emission may take place. The fluorescent signal is detected and is directly proportional to the amount of amplified nucleic acid.

Since different reporter dyes have different, and distinct emission spectra, combinations of different reporters can be strategically used in multiplex reactions. In addition to SYBR Green I, other cyanine dyes such as Cy3, or Cy5 can be used, as well as rhodamine dyes. Cy3 has a fluorescence emission at 570 nm, while Cy5 has a fluorescence emission at 670 nm. Other reporter dyes include the Alexa Fluor series of dyes, which have emission wavelengths ranging from 440 nm to 805 nm.

The three basic steps of a single round of PCR amplification are denaturation, annealing and chain extension, each optimally taking place at different temperatures (typically 94-98° C. for denaturation; 50-65° C. for annealing, and 70-80° C. for chain extension, depending on polymerase), hence the term thermocycling. Thermostable polymerases such as those described above are desirable so that their activities can be maintained during multiple cycles involving temperatures that would otherwise denature the enzyme.

The denaturation step separates the two strands of double-stranded DNA (also referred to as a DNA duplex), with each strand acting as a template in the later chain extension step in which a complete complementary strand to the template is produced. The 5′-end of a first oligonucleotide primer (typically comprising 15 to 30 nucleotides to ensure a balance of good specificity and efficient hybridization) is annealed to the 3′-end of one single stranded DNA molecule, and acts as a starting sequence for the synthesis of the new strand. A second oligonucleotide primer is at the same time annealed to the 3′-end of the other single stranded DNA molecule, and acts as a starting sequence for the synthesis of the new strand. As the two primers are together responsible for producing copies of the original DNA duplex, they are often referred to as a primer “pair”, or “pair of PCR primers”.

A DNA polymerase, using a mix of dNTPs, then synthesizes the new strand in the chain extension step, using the original single strand of DNA as its template. Since both strands of the original DNA duplex are used as templates, a round of PCR results in a doubling of the number of DNA duplexes. The number of copies thus increases exponentially with the number of rounds of amplification: after 2 rounds, four DNA duplexes are present in the sample when there was originally one DNA duplex, while after 3 rounds, 8 duplexes are present. Thus, PCR is a quick and efficient method of quickly amplifying low amounts of nucleic acid.

Multiplex PCR is a technique used for amplification of multiple, different, nucleic acid sequences of interest in a single experiment. For example, multiplex PCR may be used to screen for the presence of nucleic acid sequences of interest from multiple, different pathogens in a single reaction, such as simultaneously screening a single sample for the presence of viral nucleic acid sequences from any of SARS-COV, MERS, SARS-COV-2, influenza, and Ebola viruses. In a multiplex PCR, many different primer pairs are required, with each pair specific to a nucleic acid sequence of interest. For example, if a sample of nucleic acid was being investigated for the presence of 10 different specific nucleic acid sequences of interest (for example 10 different viruses, or 10 different genetic mutations in a patient), then at least 10 different primer pairs would be required for the multiplex PCR.

Multiplex PCR is typically performed using (a) spectral single chamber multiplexing, and (b) spatial multichamber multiplexing. The first approach uses a single chamber which simplifies fluidic design, eliminates sample splitting and avoids dilution of the target, thus potentially increasing sensitivity. However, it requires that the multiple reactions, which amplify different target nucleic acid sequences of interest, occur simultaneously. Due to competing reactions, there is a potential increase in false negatives resulting from non-specific amplification (such as primer dimerization) which may reduce the specificity and sensitivity of the assay. This approach may require a more complex optical design with more filters and lights sources, which would in turn increase costs. Furthermore, due to the practical limitations in spectral overlap of available dye indicators, only a small degree (<10) of multiplexing is possible. Spatial, multichamber multiplexing is highly desired as it allows for large degree (>100) multiplexing and avoids simultaneous competing reactions.

The present inventors have sought to develop an apparatus that addresses these challenges by providing a microfluidic device that enables a multiplexed (multitarget) rapid nucleic acid test. The present inventors have found that it is possible to provide an inexpensive apparatus which uses spatial multiplexing in a single chamber, and which allows for the test to be rapid so that a positive detection can be quickly achieved. The present inventors have found that multiplexing of a sample can be achieved by depositing different reaction reagents, for example oligonucleotide sequences that can act as PCR primers, at spatially separated locations within a single reaction chamber and performing the PCR reaction without any fluid flow. As there is no fluid flow, motion of reagents (in particular the deposited reaction reagent) and reactants is limited by diffusion and no cross reactions are therefore possible. The use of a single reaction chamber and no fluid flow for multiplex PCR enables a much more compact, and simpler device, that brings together the benefits of a single chamber set-up (simpler fluidics with no sample splitting), with the benefits of spatially separated reactions that allow for a high degree of multiplexing with no cross-reaction or interference.

In one example there is provided a microfluidic device, comprising:
a reaction chamber, wherein the reaction chamber comprises at least one reaction reagent disposed on at least one inner surface of the reaction chamber;
a heater; and
a thermally dissolvable or degradable film applied to the at least one inner surface of the reaction chamber on which the reaction reagent is disposed.

In a further example there is provided a PCR apparatus comprising a microfluidic device as described herein.

In a further example there is provided a method of producing a microfluidic device, comprising:
forming a reaction chamber in the device,
depositing on at least one inner surface of the reaction chamber a solution of a reaction reagent dissolved in a solvent;
drying the solution of reaction reagent to remove the solvent; and
applying a thermally dissolvable or degradable film to the at least one inner surface of the reaction chamber on which the reaction reagent is disposed.

In a further example there is provided a method, comprising:
introducing a test solution into a reaction chamber of a microfluidic device, wherein
   the test solution comprises a nucleic acid sample suspected of containing the nucleic acid of interest; and
   the reaction chamber of the microfluidic device comprises a first oligonucleotide of an oligonucleotide pair complementary to the nucleic acid of interest, wherein the first oligonucleotide is disposed on an inner surface of the reaction chamber and isolated from the reaction chamber by a thermally dissolvable or degradable film;
heating the test solution to dissolve and/or degrade the thermally dissolvable or thermally degradable film, exposing the first oligonucleotide to the test solution; and
subjecting the test solution to amplification by polymerase chain reaction using the oligonucleotide pair as a primer pair for the amplification.

### Microfluidic Device

Described herein is a microfluidic device. The device comprises a reaction chamber, wherein the reaction chamber comprises at least one reaction reagent disposed on at least one inner surface of the reaction chamber. The microfluidic device comprises a heater, and a thermally dissolvable or degradable film applied to the least one inner surface of the reaction chamber on which the reaction reagent is disposed.

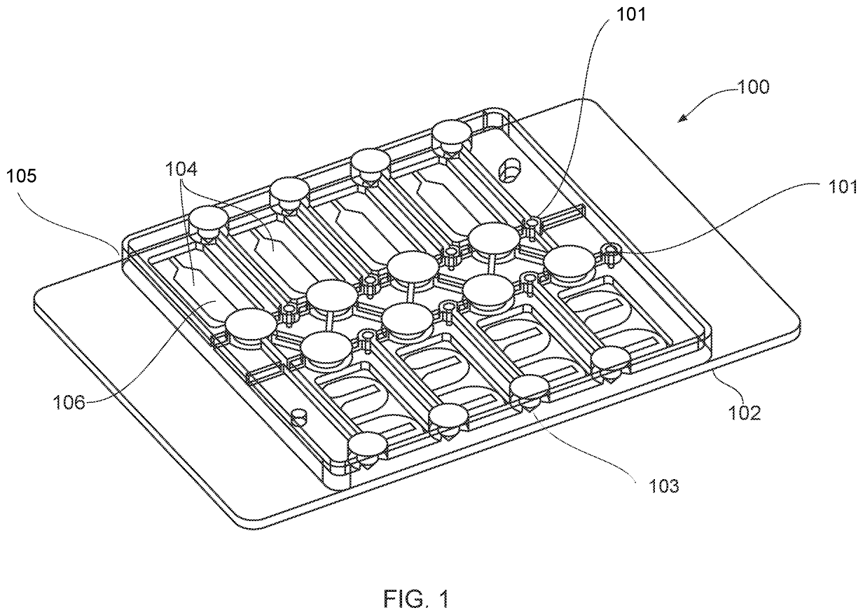

FIG. 1 shows a plan view of an example microfluidic device 100 as described herein. The microfluidic device 100 comprises a substrate 102 on top of which multiple reaction chambers 104 are formed by a microfluidic layer 105. FIG. 1 shows that the reaction chamber 104 of the microfluidic device 100 comprises at least one inner surface 106. In FIG. 1, the at least one inner surface is the base of the reaction chamber 104. The device of FIG. 1 shows eight reaction chambers: four linear, elongate reaction chambers in the top row, and four serpentine reaction chambers at the bottom row. Each reaction chamber has a dedicated inlet or fluid port 101, and a dedicated outlet (also termed a vent or exhaust) 103 formed in the microfluidic layer 105. It will be understood that the flow channel from inlet 101 to reaction chamber 104 is formed underneath the visible parts of the illustrated device

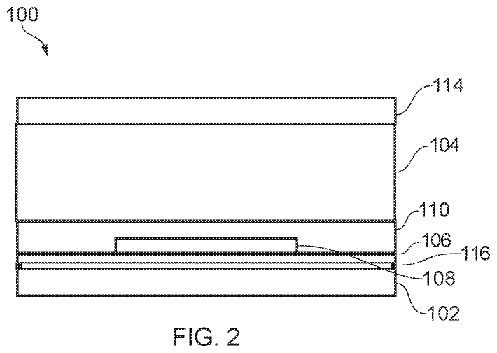

FIG. 2 is a schematic showing a side view of the microfluidic device 100 and showing features of the microfluidic device that are not visible from the view of FIG. 1. As shown in FIG. 2, the microfluidic device comprises a reaction reagent 108 disposed on a surface 106 of a reaction chamber 104 at a specific, discrete, location. In the example of FIG. 2, surface 106 is the upper surface of a substrate 102, which also includes heater 116 embedded therein. In some examples, heater 116 may be present on the upper surface of substrate 102.

In FIG. 2, a thermally dissolvable or degradable film 110 is applied to the at least one inner surface 106 of the reaction chamber 104, on which the reaction reagent 108 is at least partially disposed, so as to seal the reaction reagent 108 under the thermally dissolvable or degradable film 110, and isolate it from the rest of reaction chamber 104. In the example of FIG. 2, the reaction chamber 104 is covered and sealed by a lid 114.

In some examples, the reaction chamber 104 is a microfluidic chamber. In some examples, the microfluidic device is a single-well apparatus or a multi-well apparatus, i.e. comprises a plurality of reaction chambers (also known herein as thermocycling chambers). In some examples, each reaction chamber of the plurality of reaction chambers may have its own dedicated heater, so as to be independently controllable. In other words, each reaction chamber can have multiple reaction zones which are independently or asynchronously controllable relative to the each other. In this way, synchronous or asynchronous control of a plurality of different PCR assays each requiring a different thermocycling protocol to be performed.

In some examples, each reaction chamber may have a plurality of independently operable heaters, with each heater aligned with, for example underneath, a location at which the reaction reagent 108 is disposed. In other words, each reaction chamber can have multiple reaction zones which are independently or asynchronously controllable relative to the each other. In this way, a plurality of different PCR assays (i.e. a multiplexed PCR), each requiring a different thermocycling protocol, can be performed in a single reaction chamber. For example, different thermocycling protocols may require shorter or longer annealing times, or higher or lower annealing temperatures, based on length and content of the primers used (longer oligonucleotides, or oligonucleotides having high proportions of G:C base pairs will have higher melting temperatures, which will affect annealing of the primer to the template strand).

In some examples, the at least one inner surface 106 is the base or floor of the reaction chamber 104, as shown in FIG. 1. In some examples, the at least one inner surface is the top of a substrate on which reaction chamber 104 is disposed, for example substrate 102 as shown in FIG. 2. Substrate 102 may be formed from any material suitable for microfluidics, such as glass, silicon, SU-8 (an epoxy based photoresist material), or polycarbonate. In some examples, the heater is provided on or within a substrate 102, to provide heat to reaction chamber 104. In some examples, the substrate comprises or is a printed circuit board (PCB), and so in some examples is termed a PCB substrate. In some examples, the heater comprises one or more printed electrical traces on a substrate to provide heat to the reaction chamber. In some examples, the heater is provided above or below the plane of the microfluidic device. In some examples, the heater is embedded into a substrate on which the reaction chamber is disposed. In other examples, the heater is provided on a surface of the substrate. In some examples, the heater comprises a flat panel heater or one or more thermally conductive printed electrical traces. In some examples, the heater comprises a Peltier device, a flat panel heater in the form of a solid-state active heat pump. In some examples, the heater receives electrical power from electrically conductive wires provided on or to the microfluidic device to form an electrical circuit which supplies electrical current to the heater. Such components may be controlled by a controller located on or off the microfluidic device via control signals.

In some examples, reaction chamber 104 is defined by or provided in a microfluidic layer 105 or fluidic stack of the microfluidic device, disposed on substrate 102. In some examples, reaction chamber 104 is formed in a fluidic layer or fluidic stack by selectively etching or machining away regions of material so as to form a reaction chamber. Microfluidic layer 105 (also termed a fluidic layer or a fluidic stack) may comprise any material or combination of materials suitable for use in microfluidic devices, including polycarbonate, and cyclic olefin copolymer. As used herein, the terms “microfluidic layer”, “microfluidic stack”, “fluidic layer” or “fluidic stack” refer to the components of the microfluidic device through which one or more fluids can pass during use of the microfluidic device, for example through one or more microfluidic channels and chambers. The terms are intended to encompass multiple flow paths, for example in different levels of the layer/stack, and distinguish these flow channel-containing components from other operational modules such as electronic circuitry and sensors. Other layers present in a fluidic stack may include layers of adhesive (for example pressure-sensitive adhesives) to bond the fluidic layer to the substrate and/or bond layers of a fluid stack to each other. Suitable adhesives include pressure-sensitive adhesives, which typically comprise an elastomer based on acrylic, silicone or rubber optionally compounded with a tackifier such as a rosin ester. Convenient pressure sensitive adhesives are in the form of double-sided films or tape, such as the acrylic adhesives 200MP and 7956MP available from 3M™. In some examples, the fluidic layer is provided with one or more fluid inlets and outlets to provide a liquid such as a reaction liquid to the or each reaction chamber. In some examples, the or each reaction chamber is also provided with a vent. The presence of a vent enables unhindered flow of liquid through the reaction chamber and minimises risk of unwanted bubble formation within the reaction chamber.

In some examples, an additional barrier layer is disposed on top of the substrate, for example to protect a heater present on the top of the substrate, before the reaction reagent and thermally dissolvable or degradable film are applied. In some examples, the barrier layer may comprise, but is not limited to solder mask, Kapton®, tantalum, aluminium oxide, aluminium nitride, and silicon oxide.

As described above, in some examples a reaction reagent is disposed on at least one inner surface of the reaction chamber. In some examples, the reaction reagent may be disposed on the base or floor of the reaction chamber. In some examples, the at least one reaction reagent may also be positioned on other inner surfaces, such as peripheral walls of the reaction chamber 104 provided by microfluidic layer 105 extending from the substrate to the lid. In some examples, the at least one inner surface 104 of the reaction chamber 104 comprises multiple reaction reagents 108 and wherein each of the reaction reagents is disposed at a different location on the at least one inner surface of the reaction chamber. In some examples, each reaction reagent is a different reaction reagent. By providing different reaction reagents at different locations, a single-chamber multiplexed reaction is enabled.

Reaction reagent 108 comprises a chemical or biological material that is to be used in a reaction chemical or biological reaction to take place in reaction chamber 104. In some examples, reaction reagent 108 is introduced into reaction chamber 104 as a fluid and subsequently freeze-dried to form a freeze-dried, i.e. lyophilized, reaction reagent on a designated portion of the interior surface of reaction chamber 104. For example, a solution of reaction reagent 108 in a suitable solvent can be pipetted onto the interior surface of reaction chamber 104 during production of the microfluidic device, with the solvent subsequently being evaporated off (for example by freeze drying) to leave the deposit of reaction reagent 108 in solid form. Thus, in some examples, reaction reagent 108 comprises a freeze-dried or lyophilized reaction reagent.

In some examples, reaction reagent 108 may be a nucleic acid, for example a single strand of DNA or RNA. In some examples, the at least one reaction reagent is a single stranded oligonucleotide. In some examples, reaction reagent 108 may be an oligo (deoxy) nucleotide, that can be used as a primer in a PCR reaction. In some examples, the oligonucleotide may be a first primer of a primer pair for a PCR reaction, when the second primer of the primer pair is introduced into reaction chamber 104 separately. In some examples, reaction reagent 108 is dissolved in a suitable aqueous or organic solvent in order for it to be conveniently deposited. In some examples, reaction reagent 108 may be dissolved in water, or an aqueous buffer solution such as TE buffer (Tris-EDTA), TAE buffer (Tris-acetic acid-EDTA) or TBE buffer (Tris-borate-EDTA), and deposited by manual or robotic pipetting onto a surface of substrate 102 or other surface which will form the surface of reaction chamber 104. The component of the microfluidic device bearing the surface with deposited reaction reagent is subsequently subjected to freeze-drying (lyophilization). In the example in which reaction reagent 108 is a single strand of nucleic acid, the use of such buffers can stabilize the lyophilized nucleic acid. While reaction reagent 108 has been described with reference to nucleic acids, the present disclosure is not to be read as limited thereto, and in other examples reaction reagent 108 may be a small molecule, for example one member of a combinatorial library for which a synthetic transformation is to be simultaneously applied to all members of the combinatorial library.

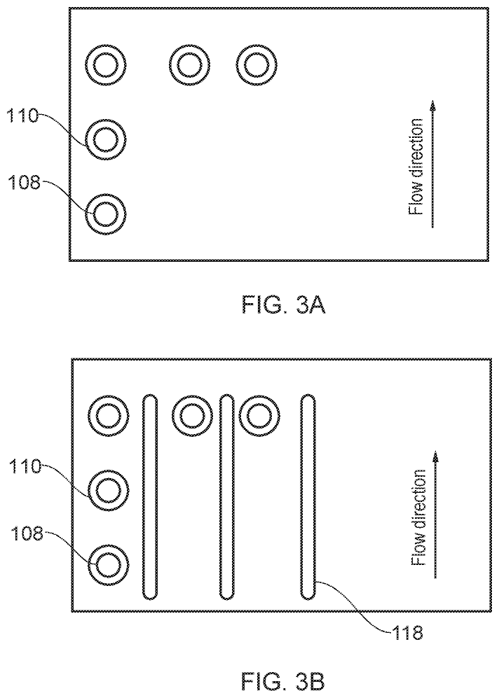

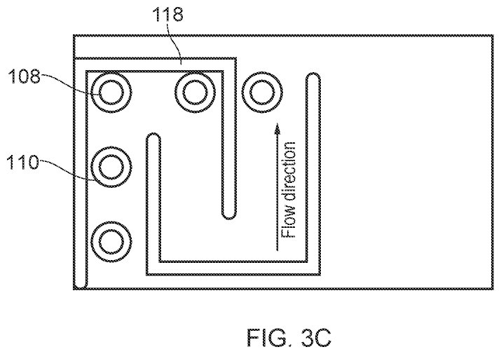

FIG. 3A shows a plan view of the microfluidic device wherein the inner surface comprises five positions (or spots) of disposed reaction reagent 108 as described herein, with each spot being spatially separated from the other spots. In some examples, the multiple reaction reagents or spots are disposed at a location of from 100 to 500 μm apart from each other, for example from 200 to 500 μm apart, for example from 300 to 500 μm apart. By spacing the reagents apart and having no fluid flow during a reaction, cross-contamination between multiple reactions is avoided. The spacing between individual spots of deposited reaction reagent will depend on the molecular size or weight of the particular reaction reagent. The larger the molecule, the less distance it will travel by simple diffusion, and the less spacing is required between adjacent spots of reaction reagent. For example, an oligonucleotide that may serve as a primer in a PCR reaction will in the absence of any forced fluid flow diffuse through a liquid at most 100 μm for a fast PCR reaction taking 10 minutes.

As can be seen in FIG. 3A, an individual film of thermally dissolvable or degradable film 110 has been applied over the individual spots of reaction reagent 108. In some examples, the thermally dissolvable or degradable film covers the reaction reagent. The film is provided in an amount sufficient to isolate reaction reagent 108 between the film and the surface of the reaction chamber. Further details on the thermally dissolvable or degradable film will be described later.

In some examples, a plurality of reaction reagents are disposed on the at least one inner surface at discrete, spaced apart locations, and wherein each reaction reagent is a different reaction reagent. In some examples, each location comprises a different reaction reagent. As different locations in the reaction chamber may comprise different reaction reagents, each location can be used for a different, specific reaction. For example, in the situation in which each reaction reagent is an oligodeoxynucleotide that can be used as a PCR primer in a PCR reaction, with different oligodeoxynucleotides being specific to different nucleic acid sequences of interest, a multiplexed PCR reaction for investigating the presence of different nucleic acid sequences of interest in a single sample can occur in the single reaction chamber. As noted above, spacing of the different reaction reagents based on their limits of diffusion avoids cross-contamination of reactions.

FIGS. 3B and 3C show other examples of microfluidic devices as described herein. In these examples, barriers 118, or flow structures, are also provided between certain reaction reagent locations, to further restrict any cross-reactivity or cross-contamination between reagent locations. Barriers 118 may be formed on the surface of the reaction chamber prior to deposition of any reaction reagent. In some examples, the barriers are integrally formed as part of the substrate on which the reaction chamber is disposed by moulding or etching (for example by laser micromachining) the substrate. In some examples, barriers 118 are deposited or affixed to the substrate in a separate manufacturing step. Barriers 118 may be formed from the same material as the substrate, or walls of the reaction chamber, or from any other suitable material such as deposited or 3D printed metal, ceramic or polymeric resin.

FIG. 3B shows an example where the barriers are formed with a parallel flow design, while FIG. 3C shows an example where the barriers are formed with a serpentine flow design. In some examples, the locations of reaction reagent in FIGS. 3A to 3C may be provided with their own individually addressable heater (not shown). In this way, individualised heating protocols can be established for each location, enabling for example different PCR thermocycling protocols to be followed, or for different thermally dissolvable or degradable films which require different temperatures for dissolving and/or degrading to be used at different locations.

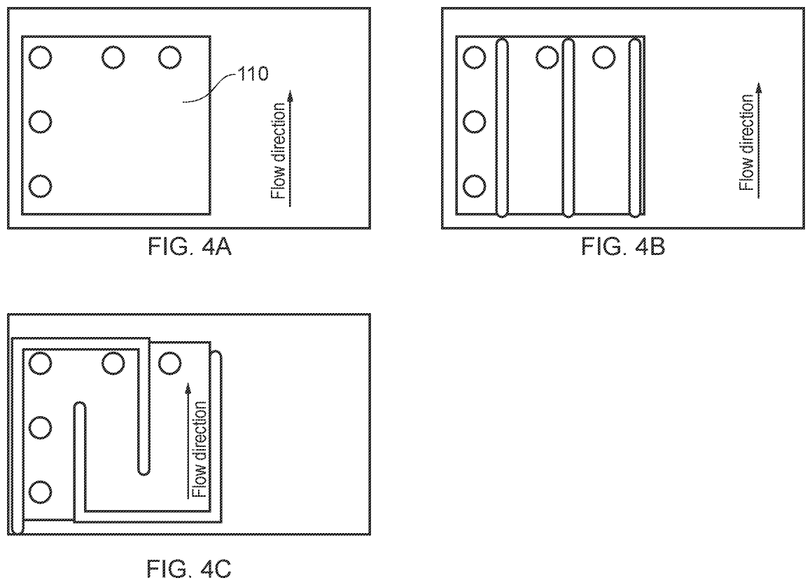

FIG. 4A shows a plan view of a microfluidic device wherein the inner surface comprises disposed reaction reagent and wherein a thermally dissolvable or degradable film has been applied to a larger area of the at least one inner surface of the reaction chamber, so as to cover or isolate all reaction reagent locations under one continuous film. As used herein, references to the at least one reagent being isolated by the thermally dissolvable or degradable film are to the reagent being in direct contact with the at least one inner surface of the reaction chamber and the thermally dissolvable or degradable film, rather than being fully encapsulated by the thermally dissolvable or degradable film in a free moving particle.

As used herein, a thermally dissolvable film is a material which is insoluble in a solution of reactants or reagents introduced into the reaction chamber until the solution is thermally actuated. In other words, a thermally dissolvable film may dissolve when in contact with a reaction solvent, for example water, and when the substrate upon which it is positioned is heated so that the temperature of the reaction solution or solvent increases.

As used herein, a thermally degradable film is a material which is stable in a solution of reactants or reagents introduced into the reaction chamber until the solution is thermally actuated. In other words, a thermally degradable film may degrade when in contact with a reaction solvent, for example water, and when the temperature of the reaction solution or solvent increases. In some examples, a thermally degradable film is degraded by the action of one or more degrading enzymes as will be described later. For example, a degrading enzyme may be disposed on the surface of the reaction chamber with the reaction reagent and isolated by the thermally degradable film.

The action of heating up the film when in contact with the reaction solvent softens the film to an extent that the degrading enzyme is no longer isolated and is redissolved in the reaction solvent and can thereby begin degrading the film. In some examples, the thermally dissolvable film is also a thermally degradable film in the presence of one or more degrading enzymes. The thermally dissolvable film may be degraded using one or more degrading enzymes after dissolving of the film. Degrading of the film prior to any thermocycling ensures that the dissolved polymer cannot indiscriminately bind to any nucleic acid strands and inhibit amplification.

In some examples, the temperature to which the reaction or test solution is heated may depend upon the composition of the film. In some examples, the reaction or test solution is heated to a temperature of from 40 to 120° C., for example from 50 to 110° C., for example from 60 to 100° C., for example from 70 to 90° C. In some examples, the dissolution temperature may be from 90° C. to 100° C. The thermally dissolvable or degradable film protects the at least one reaction reagent. In some examples, the thermally dissolvable or degradable film protects nucleic acid strands such as PCR primers, or multiple different PCR primers, from premature dissolution by an aqueous sample solution washing over the region when filling the chamber.

In some examples, the thermally dissolvable or degradable film comprises polyvinyl alcohol, polyvinyl acetate, cellulose, polyester, polyethylene terephthalate, polyurethane or combinations thereof. In some examples, the thermally dissolvable or degradable film comprises polyvinyl alcohol. In some examples, the polyvinyl alcohol comprises acetyl groups. In some examples, the polyvinyl alcohol does not comprise acetyl groups. In some examples, the polyvinyl alcohol is cross-linked. In some examples, the polyvinyl alcohol is not cross-linked. In some examples, the polyvinyl alcohol is Vinex® 1003 sold by Air Products Co, or Elvanol®, a fully hydrolysed polyvinyl alcohol sold by DuPont.

In some examples, the thermally dissolvable or degradable film comprises polyvinyl acetate, for example highly crystallized totally saponified polyvinyl acetate. In some examples, the thermally dissolvable or degradable film comprises cellulose, for example, a cellulose such as nitrocellulose.

In some examples, the thermally dissolvable or degradable film comprises one or more polymers, for example, the polymer may comprise, but is not limited to, polyester, polyethylene terephthalate and polyurethane, or combinations thereof. In some examples, one or more degrading enzymes are used to degrade these types of polymer by cleaving bonds between the monomeric units of the polymer. In some examples the degrading enzymes for degradation of polymer may include, but are not limited to cutinases (for the break-down of polyester through hydrolysis of the ester groups), polyesterases (for hydrolysis of aromatic polyesters such as polyethylene terephthalate), and enzymes incorporating polyhydroxyalkanoate binding modules (such as a polyamidase conjugated to the polyhydroxyalkanoate binding module for polyurethanes).

In some examples, the thermally dissolvable or degradable film comprises polyvinyl alcohol and the degrading enzyme involved with the degradation is polyvinyl alcohol oxidase or polyvinyl alcohol hydrogenase.

In some examples, the action of heat on the reactant solution softens and separates the film from the at least one inner surface to the extent that a degrading enzyme disposed underneath the film is then dissolved into solution and can degrade the film. In these examples, the degrading enzyme may be in solution with the reaction reagent when pipetted onto the surface or may be added as part of a separate solution which is then freeze-dried as described. The degrading enzyme may be deposited on top of, or adjacent to the reaction reagent. In some examples, a degrading enzyme is provided as part of the reaction solution that is introduced into the reaction chamber.

In some examples, the microfluidic device may be provided with a magnet in or under the substrate. In some examples, the magnet comprises a permanent magnet or an electromagnet. When used in combination with one or more magnetic beads in the test solution, with a second oligonucleotide primer of an oligonucleotide primer pair linked to a magnetic bead, the magnet can draw the bead to the surface of the reaction chamber and thus bring the oligonucleotide primer (and target nucleic acid bound to the primer through Watson-Crick base-pairing) into close proximity to the first oligonucleotide primer. In this way, any possible diffusion of the oligonucleotides can be further limited. A cleaving agent may then be used to cleave the second oligonucleotide primer from the bead to avoid any steric interference by the bead in the amplification reaction. Thus, in some examples, a cleaving reagent is also disposed with the reaction reagent. In some examples, one or both of a cleaving reagent and a degrading enzyme is disposed with the reaction reagent. The cleaving reagent may be in solution with the reaction reagent when pipetted onto the surface or may be added as part of a separate solution which is then freeze-dried as described. The cleaving reagent may be deposited on top of, or adjacent to the reaction reagent.

The nature of the cleaving reagent will depend on the initial functionalisation of the bead that allows covalent attachment of the oligonucleotide. Typically, linker groups are attached to the bead and the oligonucleotide may be covalently bound to the linker group. In some examples, the oligonucleotide is bound to the bead via a short peptidic linkage which can be cleaved enzymatically. For example, cathepsin B is a protease that cleaves a peptide bond at the C-terminal side of a dipeptide such as Phe-Arg bound to another moiety. Other enzyme-cleavable linkers can be based on β-galactoside, which can be degraded using β-galactosidase. This use of the bead is discussed further in this application in connection with the PCR method.

### PCR Apparatus

Described herein, the microfluidic device may form part of a PCR apparatus.

In some examples, the microfluidic device is in the form of a cassette, or chip, to be used in the PCR apparatus. In some examples, the microfluidic device may be a single use or disposable device. In some examples, the microfluidic device may be configured to be inserted into or received by a port in the apparatus. In some examples, the microfluidic device may be provided with one or more fluidic connections that are configured to engage with one or more corresponding fluidic connections in the apparatus, to enable fluid flow from the apparatus into the microfluidic device, for example to enable transfer of a sample injected into an injection port of the apparatus to be transferred to the reaction chamber of the microfluidic device. In other examples, the or each reaction chamber of the microfluidic device may be filled with sample prior to inserting the microfluidic device into the apparatus, for example by manual pipetting a sample solution through an inlet port such as a Luer connector or membrane valve.

In some examples, the PCR apparatus comprises an electrical interface, configured to contact an electrical interface provided on the microfluidic device. The electrical interface on the microfluidic device may be coupled to any component of the device that requires electrical current to operate. Examples of such devices include heater elements, either in flat panel form or printed conductive trace form, and actuators for controlling fluid flow within the microfluidic device. In some examples, the electrical interfaces may be multi-pin input/output off board connecters, for example 44-pin connectors that enable electrical coupling of the microfluidic device to a computer module of the PCR apparatus. Each pin of the electrical interface may provide an electrical contact to a specific component of the microfluidic device, such as the individually addressable or controllable heaters described herein. The electrical coupling of the device to the apparatus allows control signals from the computer module to be sent to the device so that electrical current can be sent to desired modules of the device.

As noted above, the PCR apparatus may comprise a computer control module. In some examples, the computer control module comprises a processor comprising hardware architecture to retrieve executable code from a data storage device or computer-readable medium and execute instructions in the form of the executable code. The processor may include a number of processor cores, an application specific integrated circuit (ASIC), field programmable gate array (FPGA) or other hardware structure to perform the functions disclosed herein. The executable code may, when executed by the processor, cause the processor to implement the functionality of one or more hardware components of the device and/or apparatus such as one or more heaters and/or one or more optical detectors. In the course of executing code, the processor may receive input from and provide output to a number of the hardware components, directly or indirectly. The computer control module may communicate with such components via a communication interface which may comprise electrical contact pads, electrical sockets, electrical pins or other interface structures. In one example, the communication interface may facilitate wireless communication.

In some examples, the computer control module facilitates the introduction of a sample into the reaction chamber, or into multiple reaction chambers. For example, the computer control module may control a series of valves and pumps in the apparatus or on the microfluidic device to direct flow of a test sample or solution to the reaction chamber.

In some examples, the computer control module may further control the processing of a sample in a reaction chamber, for example by subjecting the reaction chamber to thermocycling conditions. For example, the computer control module may control, through the output of control signals, the operation of one or more heaters to control the temperature and duration of heating within the or each reaction chamber. As a result, a sample may undergo various selected reactions, various selected heating cycles and various sensing operations under the control of the computer control module.

In some examples, the PCR apparatus comprises an optical sensor configured to obtain optical signals from the reaction chamber where thermocycling is performed. In some examples, the optical sensor is a fluorescence sensor and the optical signals are fluorescence signals. As described above, fluorescent molecules are used as reporter molecules in PCR amplification, with the fluorescence intensity proportional to the amount of amplified nucleic acid material. In some examples, the optical sensor comprises a light source and a detector, wherein the light source is for example a laser diode, or an LED, configured to emit light of a wavelength suitable to cause fluorescence of a fluorescent reporter molecule during a PCR amplification process. For example, SYBR Green I, absorbs blue light with a Amax of 497 nm, and emits green light with a Amax of 520 nm. In some examples, the detector may be a charge coupled device (CCD) or pin photodiode configured to detect the emitted fluorescent light. In some examples, the detector may be a charge coupled device (CCD) or pin photodiode to detect the emitted fluorescent light. In some examples, the optical sensor is arranged above or below the reaction chamber, for example above or below a plane in which the liquid sample is being thermocycled. In some examples, the microfluidic device is provided with an optical window or opening that allows transmission of light therethrough to an optical sensor located in the PCR apparatus but external to the microfluidic device, or within the microfluidic device itself. In some examples, the optical sensor is embedded into the lid of the microfluidic device.

### Method of Manufacturing a Microfluidic Device

In a further example there is provided a method of producing a microfluidic device, comprising:
forming a reaction chamber in the device,
providing a heater;
depositing on at least one inner surface of the reaction chamber a solution of a reaction reagent dissolved in a solvent;
drying the solution of reaction reagent to remove the solvent; and
applying a thermally dissolvable or degradable film to the at least one inner surface of the reaction chamber on which the reaction reagent is disposed.

In some examples, the reaction chamber may be provided on or in a substrate of the device, as described previously. In some examples, the reaction chamber can hold a volume of fluid of from 5 to 100 μL, for example from 10 to 75 μL, for example from 15 to 60 μL. In some examples, the method may comprise providing a heater, for example a flat panel heater, and providing the reaction chamber on top of the heater. In other examples, providing a heater may comprise affixing a heater to the underside of a substrate on which at least one reaction reagent has been disposed, or embedding the heater within the substrate. In some examples, forming the reaction chamber may comprise forming a fluidics layer or fluidic stack, and forming the reaction chamber in one or more layers of the fluidics stack by etching or micromachining the reaction chamber into the fluidics layer or fluidic stack or into a surface of the fluidics layer or fluidic stack. In some examples, forming the reaction chamber may comprise forming the reaction chamber in a fluidics layer or fluidics stack, and arranging the fluidics layer or fluidics stack comprising the reaction chamber on a substrate, with the substrate forming the floor of the reaction chamber. In some examples, the fluidics layer or fluidics stack may be bonded to the substrate by any suitable means, for example using an adhesive such as a pressure sensitive adhesive.

In some examples, the method further comprises providing barriers or flow structures to the inner surface of the reaction chamber. In some examples, providing barriers or flow structures comprises integrally forming the barriers as part of the substrate on which the reaction chamber is disposed by moulding or etching (for example by laser micromachining) the substrate. In some examples, the barriers are deposited or affixed to the inner surface of the reaction chamber in a separate manufacturing step. In some examples, the barriers may be formed from the same material as the substrate, or walls of the reaction chamber (for example the fluidics layer or fluidics stack), or from any other suitable material such as deposited metal, ceramic or polymeric resin. In some examples, the barriers may be formed by 3D printing a build material of one or more of metal, ceramic or polymeric resin onto selected regions of the substrate or inner surface of the reaction chamber.

In some examples, at least one reaction reagent is disposed on at least one inner surface of the reaction chamber. In some examples, the at least one reaction reagent is disposed on the base or the floor of the reaction chamber. In some examples, multiple reaction reagents are disposed or “spotted” onto the floor of the reaction chamber via manual or automated pipetting, or by a digital printing technique such as inkjet printing using a thermal or piezoelectric printhead. In some examples, at least one reaction reagent is disposed on a surface of a substrate that will eventually form at least one inner surface of a reaction chamber, for example once a fluidics stack in which a chamber has been has pre-formed has been arranged on the substrate.

In some examples, multiple reaction reagents are disposed or “spotted” onto the surface that will form a floor of the reaction chamber via manual or automated pipetting, or by a digital printing technique such as inkjet printing using a thermal or piezoelectric printhead. In some examples, the reaction reagents or spots are disposed at a location of from 100 to 500 μm apart, for example from 200 to 500 μm apart, for example from 300 to 500 μm apart. By spacing the reagents apart, cross-contamination between multiple reactions is avoided. In some examples, the reaction reagent is deposited or spotted in liquid form, for example as a solution in a suitable solvent, and the solvent is then removed to provide a dried reaction reagent. If the suitable solvent is a volatile solvent, then the solvent may be removed by evaporation under atmospheric pressure, or under a light vacuum. If the suitable solvent is water, or an aqueous solvent, then the solvent may be removed by freeze-drying the substrate containing the reaction reagent, resulting in the reaction reagent being in lyophilized (dried) form on the substrate.

The at least one reaction reagent may be disposed on the at least one inner surface of the reaction chamber in a manner which immobilizes it to the surface for the duration of the reaction. For example, the inner surface of the reaction chamber may comprise a surface modification that allows for covalent coupling of the reaction reagent to the surface. Nucleic acids can be immobilized on metallic surfaces by incorporating a thiol anchoring group to a terminal position of the nucleic acid.

In some examples, the method comprises applying a thermally dissolvable or degradable film to the at least one inner surface of the reaction chamber on which the reaction reagent is disposed. In some examples, a single film is applied so as to substantially cover the entire surface and all dried reaction reagents disposed thereon. Methods for applying films that substantially cover an entire surface of a substrate include screen printing, roller printing, and spin coating. In some examples, individual localised films of the thermally dissolvable or degradable film are deposited onto the substrate and reaction reagent, via pipette or a digital printing technique. In these examples, sufficient material is deposited so as to completely cover, and isolate, the reaction reagent. For example, if the reaction reagent covers an area of the surface of approximately 10 μm², then sufficient material for the film should be deposited so as to exceed this and provide an overlap on all sides, for example by depositing sufficient material to cover 20 μm².

In some examples, the step of applying the thermally dissolvable or degradable film comprises applying a solution of the thermally dissolvable or degradable material dissolved or dispersed in a solvent, and drying the solution so as to form the film of material. In some examples, the solvent used to disperse or dissolve the thermally dissolvable or degradable material comprises one or more of water, methanol, acetone or a chlorinated solvent such as chloroform or dichloromethane. In some examples, the solvent comprises a volatile solvent, which can be readily removed by simple air drying so that the film can be easily formed.

In some examples, the method isolates the at least one reagent from the reaction chamber. In some examples a cleaving reagent is provided and disposed with the at least one reaction reagent as described. In some examples, the cleaving reagent is provided to cleave a reagent from a bead as described herein. In some examples, a degrading enzyme is provided to further assist with degradation of the dissolvable or degradable film.

In some examples, the method further comprises forming a lid over the reaction chamber. In some examples, the lid forms a seal. In some examples, the lid is formed of a transparent material, to provide optical access to the reaction chamber. In some examples, the lid is formed of a material such as polycarbonate, or polypropylene, and is bonded or sealed to the fluidics layer using a pressure sensitive adhesive. In some examples, the lid forms part of the fluidics layer or fluidics stack and is provided with a fluidics interface comprising one or more fluid inlets and/or outlets, and vents.

In some examples, the method comprises providing an optical sensor configured to obtain optical signals from the reaction chamber. In some examples, the optical sensor is a fluorescent sensor. In some examples, the optical sensor is directly integrated into the microfluidic device, for example into a wall or lid of the reaction chamber or is located elsewhere in an apparatus but configured to receive signals from the reaction chamber.

### PCR Method

In some examples, there is provided a method comprising:
introducing a test solution into a reaction chamber of a microfluidic device, wherein
   the test solution comprises a nucleic acid sample suspected of containing a nucleic acid of interest; and
   the reaction chamber of the microfluidic device comprises a first oligonucleotide of an oligonucleotide pair complementary to the nucleic acid of interest, wherein the first oligonucleotide is disposed on an inner surface of the reaction chamber and isolated from the reaction chamber by a thermally dissolvable or degradable film;
heating the test solution to dissolve and/or degrade the thermally dissolvable or thermally degradable film, exposing the first oligonucleotide to the test solution; and
subjecting the test solution to amplification by polymerase chain reaction using the oligonucleotide pair as a primer pair for the amplification.

In some examples, the method is provided for performing PCR. The method may be performed on a microfluidic device as described herein, or on a PCR apparatus as described herein, which comprises the microfluidic device described herein. In some examples, the method is for detecting the presence of a nucleic acid sequence of interest in the test solution. By including in the method a primer pair that is complementary to a nucleic acid sequence of interest, and subjecting the test solution to amplification conditions, it is possible to detect the presence of the nucleic acid of interest which is suspected of being present in the nucleic acid sample. Through the amplification, low copy numbers of the nucleic acid sequence of interest (less than 10, for example less than 5 molecules) become detectable, as the number of molecules of the nucleic acid sequence of interest increases exponentially during PCR.

In some examples, the test solution comprises an aqueous solution of reactants and reagents required for PCR. In some examples, the test solution further comprises a polymerase, dNTPs and salts such as MgCl₂. Suitable polymerases include the thermostable polymerases Taq, Bst and Pfu. In some examples, the test solution comprises the four standard dNTPs, i.e. dGTP, dCTP, dATP and TTP. In some examples, the test solution also contains one or more reporter molecules that permit monitoring of the amplification by optical means. In some examples, the one or more reporter molecules comprise non-specific fluorescent dyes, such as SYBR Green, which intercalate into any double-stranded DNA, leading to an increase in fluorescence as more double-stranded DNA is produced, while other suitable reporter molecules include target-specific fluorescent reporter molecules, such as the TaqMan hydrolysis probes. The reporter molecule may be dissolved in the test solution, or may be covalently bound to a primer.

The second oligonucleotide may be disposed with the first oligonucleotide or it may be introduced into the reaction chamber by some other means. The test solution may comprise the second oligonucleotide of the oligonucleotide pair. The second oligonucleotide may be dissolved or suspended in the test solution. In some examples, the test solution comprises at least one magnetic bead and the second oligonucleotide of the oligonucleotide pair is attached to the at least one magnetic bead. In some examples, a magnet is used to draw the at least one magnetic bead to the inner surface of the reaction chamber on which the first oligonucleotide of the oligonucleotide pair is disposed. Since the second oligonucleotide primer is complementary to the target nucleic acid of interest, the target (if present) can anneal to the second oligonucleotide and also be brought to the surface of the reaction chamber by the magnetic bead. In this way, the second oligonucleotide primer is brought into close proximity to the first oligonucleotide primer. In some examples, the magnetic beads comprise of an iron oxide core, and a polymer coating. The surface of the polymer coating may also comprise functional groups which may then be covalently linked to a primer. In some examples, the bead is a colloidal magnetite (Fe₃O₄), maghemite (Fe₂O₃) or ferrite which has been surface-modified by silanisation. In some examples, the bead particle comprises a polymer core (for example polystyrene), a metal oxide shell (for example iron oxide) and a polymer coating. Examples of magnetic beads that can be covalently linked to an oligonucleotide primer include Dynabeads® from ThermoFisher.

In some examples, the test solution may be prepared by combining the nucleic acid sample, the second oligonucleotide, the dNTPs, polymerase and buffer/salts. In some examples, the test solution may be prepared by combining the nucleic acid sample, the second oligonucleotide which is covalently bound to a magnetic bead, the dNTPs, polymerase and buffer/salts, and heating the test solution to denature any double stranded DNA in the nucleic acid sample and hybridise the second oligonucleotide (covalently bound to the magnetic bead) to its complementary target nucleic acid of interest, if the target is present in the sample. In this way, not only is the second oligonucleotide primer brought to the surface of the reaction chamber by the magnet, but also the target nucleic acid sequence of interest. The magnetic bead limits diffusion of the second oligonucleotide primer, and a nucleic acid hybridised or annealed to the second oligonucleotide primer.

In some examples, the inner surface of the reaction chamber comprises a plurality of first oligonucleotides of a plurality of oligonucleotide pairs, each complementary to a different nucleic acid of interest, with each of the first oligonucleotides at a discrete, spaced apart location, and wherein the method comprises performing multiple nucleic acid amplification reactions in parallel. Accordingly, a plurality of second oligonucleotides of the plurality of the oligonucleotide pairs may be required. In these examples, which enable a multiplexed PCR analysis, each corresponding second oligonucleotide may be bound to a separate magnetic bead as described. Alternatively, the second oligonucleotide or the plurality of second oligonucleotides may be disposed with their respective first oligonucleotides and isolated by the thermally dissolvable or degradable film. Thus, references to the first oligonucleotide being exposed to the test solution refer also to the second oligonucleotide being exposed to the test solution, when that is also isolated under the film. It is also possible for the second oligonucleotide to be present in the test solution without being bound to a magnetic bead, or to be introduced into the reaction chamber separately to the test solution (whether bound to a magnetic bead or not).

During the method, the test solution may be flowed into a reaction chamber or into each one of multiple reaction chambers. In some examples, once the test solution has completely filled the reaction chamber to the exclusion of any air bubbles which can be expelled via a vent, no further fluid flow occurs in the reaction chamber. That is, in some examples, once the test solution has been introduced into the reaction chamber, the method is performed in the absence of any fluid flow into or through the reaction chamber. Once the test solution has been introduced into the reaction chamber, the at least one inner surface of the reaction chamber may then be heated (for example by providing a current to a PCB forming at least part of the substrate) to a temperature which causes the thermally dissolvable or degradable film to be dissolve and/or degrade as described, so that the first oligonucleotide primer can be exposed. In some examples, the temperature to which the test solution is to be heated may depend upon the composition of the film.

In some examples, the temperature to which the test solution is to be heated may be from 40 to 120° C., for example from 50 to 110° C., for example from 60 to 100° C., for example from 70 to 90° C. In some examples, the temperature may be from 90° C. to 100° C. The first oligonucleotide will then be exposed, and may then solubilise into the solution in the same location where it was originally disposed. As there is minimum or zero flow in the reaction chamber, the motion of the nucleic acid material (the two oligonucleotides serving as primers, and the larger nucleic acid of interest) is limited because of diffusion. In some examples, the method then comprises subjecting the test solution to amplification by polymerase chain reaction using the oligonucleotide pair as a primer pair for the amplification. In some examples, subjecting the test solution to amplification by polymerase chain reaction comprises thermocycling the test solution in the reaction chamber.

In some examples, a cooling block may be placed under the heater in order to accelerate the cooling step of thermocycling. In some examples, the cooling block may comprise a Peltier element or Peltier device, which transfers heat from one side of the device to the other, with consumption of electrical energy, depending on the direction of the current.

In some examples, the inner surface of the reaction chamber comprises a plurality of first oligonucleotides of the plurality of oligonucleotide pairs, each at a discrete, spaced apart location. In some examples, each oligonucleotide pair is complementary to a different nucleic acid of interest. As different locations in the reaction chamber comprise different oligonucleotides which can act as primers for different target nucleic acids of interest, simultaneous tests for the presence of different targets can occur. In some examples, the individual locations are spaced apart to avoid cross-contamination. In some examples, the individual locations are spaced from 100 μm to 1000 μm apart, in some examples from 200 to 800 μm apart, in some examples 300 to 600 μm apart, and in some examples 500 μm apart.

The first oligonucleotide or each first oligonucleotide of a plurality of oligonucleotide pairs may be immobilized on the inner surface of the reaction chamber, remaining immobilized after the thermally dissolvable or degradable film has been removed. Immobilizing the oligonucleotide ensures that there is no diffusion whatsoever and constrains the reaction to that location. However, to ensure that the PCR reaction is not impeded by the surface, the second oligonucleotide or each second oligonucleotide of a plurality of oligonucleotide pairs may be in solution, and not bound to a magnetic bead.

In some examples, one or both of a degrading enzyme and a cleaving reagent is also isolated with the second oligonucleotide by the thermally dissolvable or degradable film and wherein, after the heating step, the cleaving reagent is released and cleaves the second primer from the at least one bead and/or the degrading enzyme is released and degrades the film. In some examples, a cleaving reagent is also disposed along with the first primer as described herein. Upon softening, or dissolving, of the thermally dissolvable or degradable film, the cleaving reagent may also be released. This cleaving reagent may then cleave the second primer from the bead, allowing the PCR reaction to occur in solution and thus be more efficient than a surface-based reaction. In some examples, the second oligonucleotide may be cleaved using external influence, for example, using heat or UV light, instead of enzymatically. UV-cleavable linkers include the nitrobenzyl linker.

In some examples, the test solution has a volume of less than 100 μL, for example less than 50 μL, for example less than 25 μL, for example less than 10 μL, for example about 5 μL. In some examples, the test solution has a volume of greater than 5 μL, for example greater than 10 μL, for example greater than 25 μL, for example greater than 50 μL, for example about 100 μL.

In some examples, the test solution comprises a nucleic acid sample obtained from a subject. In some examples, the nucleic acid sample may comprise a nucleic acid for analysis and is to be amplified in a method as described herein. In some examples, the nucleic acid sample may comprise a plurality of nucleic acids for analysis which are to be amplified in a method as described herein. In some examples, the test solution is suspected of comprising a one or a plurality of nucleic acid sequences of interest. In some examples, the nucleic acid sample is obtained from one or more of a blood sample, a tissue sample, a saliva sample or mucosal sample. In some examples, the nucleic acid sample is obtained using a swab. In some examples, the nucleic acid sample is isolated from the bodily fluid or tissue via which it was obtained. In some examples, the nucleic acid sample is not isolated from the bodily fluid or tissue via which it was obtained. In some examples, the nucleic acid sample obtained from a subject is incorporated into a test solution with or without any isolation or preparation. In some examples, the nucleic acid sample obtained from a subject is dissolved or dispersed in an aqueous solution, thus forming a test solution.

In some examples, a second oligonucleotide of an oligonucleotide pair complementary to a nucleic acid of interest which is suspected of being in the nucleic acid sample is present in the test solution when the nucleic acid sample is dissolved or dispersed in the solution, or is added to the solution after the nucleic acid sample has been dissolved or dispersed. The second oligonucleotide may be dissolved or suspended in the test solution before or after the nucleic acid sample has been dissolved or dispersed, or the second oligonucleotide may be mixed with the nucleic acid sample before being added to the test solution. In some examples, a second oligonucleotide of an oligonucleotide pair complementary to a nucleic acid of interest which is suspected of being in the nucleic acid sample is introduced into the reaction chamber of the microfluidic device separately to the test solution. For example, the second oligonucleotide of the oligonucleotide pair may also be disposed on the inner surface of the reaction chamber and be isolated under the thermally dissolvable or degradable film, or it may be introduced as a separate solution before or after the test solution has been introduced.

In some examples, a polymerase, and mix of dNTPs may also be added to the test solution before or after the nucleic acid sample has been dissolved or dispersed. A PCR “Master Mix” may be added to the test solution before or after the nucleic acid sample has been dissolved or dispersed. A PCR Master Mix is a mixture of PCR reagents, already at optimized concentrations, which can be readily aliquoted and added to the test solution. The Master Mix usually comprises the DNA elongation enzyme (e.g. a polymerase), the dNTPs, MgCl₂ as an enzyme co-factor (although other co-factors, such as MgSO₄ may be used with certain enzymes), all dissolved in an aqueous buffer. The Master Mix may also include a reporter molecule, such as a fluorescent dye as described herein. The LightCycler® 480 SYBR Green I Master Mix includes a polymerase, co-factor, dNTPs and SYBR Green I in a buffered solution, meaning that only the nucleic acid sample (and, if appropriate, a primer) need to be added. However, the reporter molecule may also be added separately.

In some examples, the nucleic acid is subjected to amplification conditions by PCR by thermocycling the test solution for up to 40 cycles. In some examples, the denaturation step may take from 0.1 to 3 seconds, and in some examples, 0.5 seconds. In some examples, the annealing step may take from 0.1 to 3 seconds, and in some examples, 0.5 seconds. In some examples the extension step may take from 1 to 60 seconds, and in some examples, from 5 to 10 seconds.

A fluorescence detector is used to detect and measure the fluorescence level at each position of a first oligonucleotide. In some examples, either during or at the end of thermocycling, a fluorescence detector is used to detect and measure the fluorescence level at each position of a first oligonucleotide. In some examples, the fluorescence sensor detects and measures the fluorescence level after each thermocycle, or after 5 thermocycles, or after 10 thermocycles, or any number of cycles as required. If a nucleic acid of interest is present in the sample, it will be amplified through the thermocycling, using the complementary oligonucleotide primer pair, one oligonucleotide of which was disposed at a specific location or position in a surface of the reaction chamber. Since the amplification of that particular nucleic acid of interest takes place at the specific location or position in the reaction chamber, measurement of any presence or increase in fluorescence at that position is an indication that the nucleic acid of interest was present in the sample or test solution. The sooner that a positive result (via fluorescence detection) confirms that a nucleic acid of interest is present in a test solution, the quicker the overall test time.

The present invention enables a simple, rapid point-of-care diagnostics array device that can accurately and simultaneously screen for multiple nucleic acid sequences of interest.

While the apparatus, methods and related aspects have been described with reference to certain examples, it will be appreciated that various modifications, changes, omissions, and substitutions can be made without departing from the spirit of the disclosure. It is intended, therefore, that compositions, methods and related aspects be limited only by the scope of the following claims. Unless otherwise stated, the features of any dependent claim can be combined with the features of any of the other dependent claims, and any other independent claim.

1. A microfluidic device, comprising:
a reaction chamber, wherein the reaction chamber comprises at least one reaction reagent disposed on at least one inner surface of the reaction chamber;
a heater; and
a thermally dissolvable or degradable film applied to the at least one inner surface of the reaction chamber on which the reaction reagent is disposed.

2. The microfluidic device according to claim 1, wherein the at least one inner surface of the reaction chamber comprises multiple reaction reagents and wherein each of the reaction reagents is disposed at a different location on the at least one inner surface of the reaction chamber.

3. The microfluidic device according to claim 2, wherein each reaction reagent is a different reaction reagent.

4. The microfluidic device according to claim 1, wherein the at least one reaction reagent is a single stranded oligonucleotide.

5. The microfluidic device according to claim 1, wherein the thermally dissolvable or degradable film comprises polyvinyl alcohol, polyvinyl acetate, cellulose, polyester, polyethylene terephthalate, polyurethane or combinations thereof.

6. The microfluidic device according to claim 1, wherein the thermally dissolvable or degradable film isolates the reaction reagent from the reaction chamber.

7. The microfluidic device according to claim 1, wherein one or both of a cleaving reagent and a degrading enzyme is disposed with the reaction reagent.

8. The microfluidic device according to claim 1, wherein the heater comprises a flat panel heater or one or more thermally conductive printed electrical traces.

9. A method, comprising:
introducing a test solution into a reaction chamber of a microfluidic device, wherein
the test solution comprises a nucleic acid sample suspected of containing a nucleic acid of interest; and
the reaction chamber of the microfluidic device comprises a first oligonucleotide of an oligonucleotide pair complementary to the nucleic acid of interest, wherein the first oligonucleotide is disposed on an inner surface of the reaction chamber and isolated from the reaction chamber by a thermally dissolvable or degradable film;
heating the test solution to dissolve and/or degrade the thermally dissolvable or thermally degradable film, exposing the first oligonucleotide to the test solution; and
subjecting the test solution to amplification by polymerase chain reaction using the oligonucleotide pair as a primer pair for the amplification.

10. The method according to claim 9, wherein the inner surface of the reaction chamber comprises a plurality of first oligonucleotides of a plurality of oligonucleotide pairs, each complementary to a different nucleic acid of interest, with each of the first oligonucleotides at a discrete, spaced apart location, and wherein the method comprises performing multiple nucleic acid amplification reactions in parallel.

11. The method according to claim 9, wherein, once the test solution has been introduced into the reaction chamber, the method is performed in the absence of any fluid flow into or through the reaction chamber.

12. The method according to claim 9, wherein the test solution comprises a second oligonucleotide of the oligonucleotide pair.

13. The method according to claim 9, wherein the test solution comprises at least one magnetic bead and a second oligonucleotide of the oligonucleotide pair is attached to the at least one magnetic bead.

14. The method according to claim 13, wherein a magnet is used to draw the at least one magnetic bead to the inner surface of the reaction chamber on which the first oligonucleotide of the oligonucleotide pair is disposed.

15. The method according to claim 9, wherein one or both of a degrading enzyme and a cleaving reagent is also isolated from the reaction chamber with the first oligonucleotide by the thermally dissolvable or degradable film and wherein, after the heating step, the cleaving reagent is released and cleaves the first primer from the at least one bead and/or the degrading enzyme is released and degrades the film.
